(19) United States
(12) Patent Application Publication
(10) Pub. No.: US 2026/0125709 A1
(43) Pub. Date: May 7, 2026

(54) METHOD FOR TARGETED ALTERATION OF DUPLEX DNA

(71) Applicant: Keygene N.V., Wageningen (NL)

(72) Inventor: Paul BUNDOCK, Wageningen (NL)

(73) Assignee: Keygene N.V., Wageningen (NL)

(21) Appl. No.: 19/209,115

(22) Filed: May 15, 2025

Related U.S. Application Data
(62) Division of application No. 16/449,326, filed on Jun. 21, 2019, now Pat. No. 12,331,309.

(51) Int. Cl. C12N 15/90; C12N 9/22; C12N 15/10; C12N 15/11

(52) U.S. Cl. CPC C12N 15/90; C12N 9/22; C12N 15/102; C12N 15/11; C12N 2310/20; C12N 2800/80

(57) ABSTRACT

The current invention relates to methods of targeted genetic alteration in plant cells, as well as to plant cells and plants thus obtained. In the method, a Cpf1 protein and a crRNA is employed to provide for targeted alteration, in particular increased biallelic alteration, of a DNA duplex with increased efficacy.

### FIELD OF THE INVENTION

The invention relates to the field of genome or gene editing tools.

### BACKGROUND OF THE INVENTION

The process of deliberately creating alterations in the genetic material of living cells generally has the goal of modifying one or more genetically encoded biological properties of that cell, or of the organism of which the cell forms part or into which it can regenerate. These changes can take the form of deletion of parts of the genetic material, addition of exogenous genetic material, or changes in the existing nucleotide sequence of the genetic material.

Methods of altering the genetic material of eukaryotic organisms have been known for over 20 years, and have found widespread application in plant, human and animal cells and micro-organisms for improvements in the fields of agriculture, human health, food quality and environmental protection.

The most common methods consist of adding exogenous DNA fragments to the genome of a cell, which will then confer a new property to that cell or its organism over and above the properties encoded by already existing genes (including applications in which the expression of existing genes will thereby be suppressed). Although many such examples are effective in obtaining the desired properties, these methods are nevertheless not very precise, because there is no control over the genomic positions in which the exogenous DNA fragments may be inserted (and hence over the ultimate levels of expression), and because the desired effect will have to manifest itself over the natural properties encoded by the original and well-balanced genome.

On the contrary, methods of genome editing that will result in the addition, deletion or conversion of nucleotides in predefined, i.e. targeted, genomic loci will allow the precise modification of the genome, for example in existing genes.

A large part of targeted genome editing described in the literature has been performed using zinc finger nucleases (ZFNs). ZFNs have been used to modify endogenous genes in a wide range of organisms and cell types. Several types of genomic alterations can be introduced with ZFNs including point mutations, deletions, insertions, inversions, duplications, and translocations, thus providing researchers with unprecedented tools to perform genetic manipulations.

In the more recent years, transcription activator-like effector nucleases (TALENs) have rapidly emerged as an alternative to ZFNs for genome editing and introducing targeted double-strand breaks (DSBs), i.e. in which both strands of the DNA duplex are severed. TALENs are similar to ZFNs and comprise a non-specific FokI nuclease domain fused to a customizable DNA-binding domain. This DNA-binding domain is composed of highly conserved repeats derived from transcription activator-like effectors (TALEs), which are proteins secreted by Xanthomonas bacteria to alter transcription of genes in host plant cells.

In more detail, TALEs consist of a number of repeating protein domains, each of which is able to specifically recognize and bind to one of the 4 DNA nucleotides (A,T,G,C). The domains specific for each nucleotide have been identified and arrays of these domains which have high binding affinity for any DNA sequence can be produced (Christian, 2010, Genetics 186:757-761; Cermak et al., 2011, Nucleic Acids Res 39:e82; Bogdanove and Voytas, 2011, Science 333:1843-1846; Boch, 2011, Nature Biotechnology 29:135-136). These arrays are then fused to the nuclease domain of FokI to create a TALEN and, similar to ZFN, two TALEN proteins are used to induce a DNA DSB in the target DNA duplex. Several papers have described the use of TALENs to create mutations at the target sequence (Curtin (2012) The Plant Genome, 5, 42-50). Joung et al. (Nat Rev Mol Cell Biol. (2013) 14(1): 49-55. doi: 10.1038/nrm3486A) reviewed and compared various techniques employing TALENs in targeted genome editing.

Recently, another novel method for targeted genome editing has been reported. CRISPRs (Clustered Regularly Interspaced Short Palindromic Repeats) are loci containing multiple short direct repeats and are found in 40% of the sequenced bacteria and 90% of sequenced archaea.

The CRISPR repeats form a system of acquired bacterial immunity against genetic pathogens such as bacteriophages and plasmids. When a bacterium is challenged with a pathogen, a small piece of the pathogen genome is processed by CRISPR associated proteins (Cas) and incorporated into the bacterial genome between CRISPR repeats. The CRISPR loci are then transcribed and processed to form so called crRNA's which include approximately 30 nucleotides of sequence identical to the pathogen genome. These RNA molecules form the basis for the recognition of the pathogen upon a subsequent infection and lead to silencing of the pathogen genetic elements through either a RNAi-like process or direct digestion of the pathogen genome.

The CRISPR-Cas systems of bacterial and archaeal adaptive immunity show extreme diversity of protein composition and genomic loci architecture. The CRISPR-Cas system has more than 50 gene families and there are no strictly universal genes indicating fast evolution and extreme diversity of loci architecture. The Cas9 protein is an example of a Cas protein of the type II CRISPR/Cas system and forms an endonuclease, when combined with the crRNA and a second RNA termed the trans-activating crRNA (tracrRNA), which targets the invading pathogen DNA for degradation by the introduction of DNA double strand breaks (DSBs) at the position in the pathogen genome defined by the crRNA. Jinek et al. (2012, Science 337:816-820) demonstrated that a single chain chimeric guide RNA (herein “sgRNA) produced by fusing an essential portion of the crRNA and tracrRNA was able to form a function endonuclease in combination with the Cas protein.

Next to the CRISPR/CAS9 system, a new CRISPR/CAS system has recently been described, the Clustered Regularly Interspaced Short Palindromic Repeats from Prevotella and Francisella 1 or CRISPR/Cpf1. Cpf1 genes are associated with the CRISPR locus and encode an endonuclease that uses a crRNA to target DNA. Cpf1 is a smaller and simpler endonuclease than Cas9, which may overcome some of the CRISPR/Cas9 system limitations. Cpf1 is a single RNA-guided endonuclease lacking tracrRNA, and it utilizes a T-rich protospacer-adjacent motif. Cpf1 cleaves DNA via a staggered DNA double-stranded break (Zetsche et al (2015) Cell 163 (3): 759-771).

Despite these recent advances in understanding mechanisms of targeted DNA alteration, targeted alteration in plant material is still not always successful or efficient. Indeed, available methodology is often optimized for animal, in particular human, cell material and is not always successful or efficient when applied specifically to plant cells. Additionally, the prior art methodology provides genetically manipulated organisms, which, in some embodiments, is not preferred in the plant field. Thus, there is a need for new methods of providing plant cells wherein a targeted alteration has been introduced with a system and protocol specifically designed for such plant cells. Such methods of targeted alteration of DNA in a plant cell may, preferably, be successfully applied on various plant cells and with a suitable efficiency in comparison to methods known in the art.

In light of this, new methods for targeted alteration of DNA in plant cells, and for providing plant cells and plants wherein a targeted alteration has been introduced, would be highly desirable. In particular, there is a clear need in the art for reliable, efficient, reproducible and in particular targeted methods that allow for efficient targeted alteration of a DNA molecule in a plant cell. Accordingly, the technical problem underlying the present invention can be seen in the provision of methods for complying with any of the aforementioned needs. The technical problem is solved by the embodiments characterized in the claims and disclosure below.

### SUMMARY OF THE INVENTION

In a first aspect, the present disclosure provides a method of targeted alteration of duplex DNA in a plant, wherein the first DNA strand of the duplex DNA comprises a target sequence and the second DNA strand of the duplex DNA comprises a sequence complement to the target sequence, the method comprising:
(a) providing plant cells or plant protoplasts comprising said duplex DNA; and
(b) exposing said duplex DNA in said cells or protoplasts to:
   a Cpf1 protein; and
   a crRNA comprising a guide sequence for targeting said Cpf1 protein to the site of the duplex DNA comprising the target sequence.

Said duplex DNA may be exposed to said Cpf1 by introducing into the plant protoplast a nucleic acid construct for the transient expression of said Cpf1.

Said duplex DNA may be exposed to said crRNA by introducing into the plant protoplast a nucleic acid construct for transient expression of said crRNA.

In an embodiment, said duplex DNA is exposed to said Cpf1 and crRNA by introducing into the plant protoplast a nucleic acid construct for the transient expression of said Cpf1 and said crRNA.

In an embodiment, the nucleotide sequence encoding said crRNA is operably linked to a polIII promoter.

In an embodiment, said construct for the transient expression of said Cpf1 comprises a nucleotide sequence encoding Cpf1 that is operably linked to a constitutive 35S promoter.

In an embodiment, in step (b) the duplex DNA is exposed to said Cpf1 and crRNA by introduction thereof, or of a nucleic acid construct encoding the same, using polyethylene glycol mediated transformation.

In a preferred embodiment, the nucleotide sequence encoding Cpf1 is codon-optimized for expression in plant cells or plant protoplast.

In an embodiment, the method taught herein further comprises a step of synchronizing the cell cycle phase of the cell or protoplast, preferably before and/or during performing step (b), preferably wherein synchronizing is performed by contacting the cell or protoplast with a synchronizing agent.

In an embodiment, the step of synchronizing the cell phase synchronizes the protoplast in the S-phase, the M-phase, the G1 and/or G2 phase of the cell cycle.

In an embodiment, two or more crRNAs are used.

In an embodiment, at least one targeted alteration is introduced in the duplex DNA, preferably wherein the alteration comprises the insertion, deletion or modification of at least one base pair.

In an embodiment, the alteration comprises the deletion of at least one base pair and the insertion of at least one base pair.

In an embodiment, the at least one targeted alteration is biallelic.

In an embodiment, the method taught herein further comprises the step of regenerating a plant cell or plant from the plant protoplast, wherein the plant cell, plant or progeny thereof comprises the at least one targeted alteration.

Finally, the present disclosure teaches a plant, plant part, seed, or plant cell obtainable by the method taught herein, wherein the plant, plant part, seed, or plant cell is modified compared to a control plant, plant part, seed, or plant cell, and wherein said control plant, plant part, seed, or plant cell is a plant, plant part, seed, or plant cell before the at least one targeted alteration was introduced by the method taught herein.

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

The singular terms “a,” “an,” and “the” include plural referents unless the context clearly dictates otherwise. Thus, for example, reference to “a cell” includes a combination of two or more cells, and the like.

The term “and/or” refers to a situation wherein one or more of the stated cases may occur, alone or in combination with at least one of the stated cases, up to with all of the stated cases.

The term “comprising” is construed as being inclusive and open ended, and not exclusive. Specifically, the term and variations thereof mean the specified features, steps or components are included. These terms are not to be interpreted to exclude the presence of other features, steps or components.

The term “chimeric gene” refers to any gene which is not normally found in nature in a species, in particular a gene in which one or more parts of the nucleic acid sequence are not associated with each other in nature. For example, the promoter is not associated in nature with part or all of the transcribed region or with another regulatory region. The term “chimeric gene” is understood to include expression constructs in which a promoter or transcription regulatory sequence is operably linked to one or more coding sequences.

“Codon-optimized”: this term refers to one or more replacement(s) of codon of a nucleic acid from a first organism (for example a bacterium) with codon more frequently used in a second, different, organism (for example a plant), to adapt and optimize (gene) expression in the second organism.

“Construct” or “nucleic acid construct” or “vector”: this refers to a man-made nucleic acid molecule resulting from the use of recombinant DNA technology and which is used to deliver exogenous DNA into a host cell, often with the purpose of expression in the host cell of a DNA region comprised on the construct. The vector backbone of a construct may for example be a plasmid into which a (chimeric) gene is integrated or, if a suitable transcription regulatory sequence is already present (for example a (inducible) promoter), only a desired nucleic acid sequence (e.g. a coding sequence) is integrated downstream of the transcription regulatory sequence. Vectors may comprise further genetic elements to facilitate their use in molecular cloning, such as e.g. selectable markers, multiple cloning sites and the like.

“Expression”: this refers to the process wherein a DNA region, which is operably linked to appropriate regulatory regions, particularly a promoter, is transcribed into an RNA, which is capable of being translated into a protein or peptide.

“Plant”: this includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, grains and the like. Non-limiting examples of plants include crop plants and cultivated plants, such as barley, cabbage, canola, cassava, cauliflower, chicory, cotton, cucumber, eggplant, grape, hot pepper, lettuce, maize, melon, oilseed rape, potato, pumpkin, rice, rye, sorghum, squash, sugar cane, sugar beet, sunflower, sweet pepper, tomato (e.g. Solanum lycopersicum), water melon, wheat, and zucchini.

“Sequence” or “Nucleic acid sequence”: This refers to the order of nucleotides of, or within a nucleic acid. In other words, any order of nucleotides in a nucleic acid may be referred to as a sequence of nucleic acid sequence. Likewise, a “target sequence” is to denote an order of nucleotides within a nucleic acid that is to be targeted, i.e. wherein an alteration is to be introduced. Within the context of the current invention a first nucleic acid sequence may be comprised within or overlap with a further nucleic acid sequence. For example, the targets sequence is an order of nucleotides comprised by a first strand of a DNA duplex.

“Guide sequence” is to be understood herein as the section of the guide RNA (which preferably is a crRNA or sgRNA) which is for targeting the guide RNA to the target sequence.

In the context of formation of a CRISPR complex, the term “target sequence” refers to a sequence to which a guide sequence is designed to target, e.g. have complementarity, where hybridization between a target sequence and a guide sequence promotes the formation of a CRISPR complex. The guide RNA may be sgRNA (e.g. in case of Cas9) comprising a tracrRNA sequence or crRNA (e.g. in case of Cpf1) lacking a tracrRNA sequence. A target sequence may comprise any polynucleotide, such as DNA or RNA polynucleotides and is comprised within a target locus of interest. In some embodiments, a target sequence is located in the nucleus or cytoplasm of a cell. The target sequence may be any DNA or RNA sequence. In some embodiments, the target sequence may be a sequence within a RNA molecule selected from the group consisting of messenger RNA (mRNA), pre-mRNA, ribosomaal RNA (rRNA), transfer RNA (TRNA), micro-RNA (miRNA), small interfering RNA (siRNA), small nuclear RNA (snRNA), small nucleolar RNA (snoRNA), double stranded RNA (dsRNA), non coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably, the guide sequence is 10-30 or 15-25 nucleotides long.

The term “a nucleic acid-targeting complex” or “CRISPR complex” as used herein refers to a complex of a guide RNA hybridized to a target sequence and complexed with one or more Cpf1 proteins. Formation of a nucleic acid-targeting complex results in cleavage of both DNA or RNA strands in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g. within 1, 2, 3, 4, 5, 6, 7, 8, 9, 10, 20, 50, or more base pairs from the target sequence, wherein the target sequence is comprised within a target locus of interest).

### DETAILED DESCRIPTION OF THE INVENTION

In a first aspect, the present disclosure provides a method for targeted alteration of duplex DNA, e.g. at a target locus of interest, in a plant, wherein the first DNA strand of the duplex DNA comprises a target sequence and the second DNA strand of the duplex DNA comprises a sequence complementary to the target sequence, the method comprising the steps of:
(a) providing plant cells or plant protoplasts comprising said duplex DNA; and
(b) exposing said duplex DNA in said protoplasts to
   a Cpf1 protein; and
   a guide RNA (crRNA) comprising a guide sequence for targeting said Cpf1 to the site of the duplex DNA, e.g., at the target locus of interest, comprising the target sequence.

In an embodiment, the targeted alteration comprises the introduction of a strand break, such as a double-strand break.

The duplex DNA may be present at a target locus of interest. The duplex DNA may be comprised in a DNA molecule within a cell. The cell may be a plant cell, or a plant protoplast. The plant cell or plant protoplast may be of a crop plant such as a monocot or dicot or of a crop or grain plant such as cassava, corn, sorghum, soybean, wheat, oat or rice. The plant cell may also be of an alga, tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; plants of the genus Solanum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc). Preferably, the plant cell is Solanum lycopersicum plant cell.

The invention provides a method of modifying a target locus of interest, the method comprising delivering to said locus a non-naturally occurring or engineered composition comprising a Cpf1 protein and one or more nucleic acid components, for example a crRNA, wherein the Cpf1 protein forms a complex with the one or more nucleic acid components and upon binding of the said complex to the locus of interest the Cpf1 protein induces the modification of the target locus of interest. In a preferred embodiment, the modification is the introduction of a double-strand break.

The target locus of interest may be a genomic or epigenomic locus of interest. The complex may be delivered with multiple crRNAs for multiplexed use. More than one Cpf1 protein(s) may be used. Any Cpf1 protein, including orthologues or engineered Cpf1 proteins that recognize different PAM sequences, may be used.

According to the invention, any type of plant cell may be used in the method as long as the plant cells allows the exposure of the DNA duplex to the site-specific nuclease, the single-stranded oligonucleotide and, in some embodiments, the guide RNA (crRNA). However, in a preferred embodiment the plant cell is a plant protoplast. The skilled person is aware of methods and protocols for preparing and propagation plant protoplasts, see for example Plant Tissue Culture (ISBN: 978-0-12-415920-4, Roberta H. Smith). The plant protoplasts for use in the method of the current invention can be provided using common procedures (e.g. using cellulases and pectinase) used for the generation of plant cell protoplasts.

Plant cell protoplasts systems have for example been described for tomato, tobacco and many more (Brassica napus, Daucus carota, Lactucca sativa, Zea mays, Nicotiana benthamiana, Petunia hybrida, Solanum tuberosum, Oryza sativa). The present invention is generally applicable to any protoplast system, including those, but not limited to, the systems described in any one of the following references: Barsby et al. 1986, Plant Cell Reports 5(2): 101-103; Fischer et al. 1992, Plant Cell Rep. 11(12): 632-636; Hu et al. 1999, Plant Cell, Tissue and Organ Culture 59:189-196; Niedz et al. 1985, Plant Science 39:199-204; Prioli and Söndahl, 1989, Nature Biotechnology 7:589-594; S. Roest and Gilissen 1989, Acta Bot. Neerl. 38(1): 1-23; Shepard and Totten, 1975, Plant Physiol. 55:689-694; Shepard and Totten, 1977, Plant Physiol. 60:313-316, which are incorporated herein by reference.

The Cpf1 protein may be any Cpf1 protein known in the art or yet to be discovered. In an embodiment, the Cpf1 protein may be the Cpf1 protein of Acidaminococcus or a Cpf1 protein derived from Acidaminococcus. Said Acidaminococcus Cpf1 protein or said protein derived from Acidaminococcus may be codon-optimized for expression in plants. The skilled person is well-acquainted with methods of codon-optimizing expression of foreign proteins in plants.

In an embodiment, said Cpf1 protein is a protein having an amino acid sequence as shown in FIG. 1, or a variant thereof having at least 70%, such as at least 75%, 80%, 85%, 90%, 95%, 96%, 97%, 98%, or 99% sequence identity to the protein having an amino acid sequence as shown in FIG. 1.

In a preferred embodiment of the method of the invention the duplex DNA is exposed to the Cpf1 protein by introducing into the plant cell a nucleic acid construct for expression of the Cpf1 protein in the plant cell.

The methods of the invention do not depend on a particular method for introducing the Cpf1 protein into the plant cell. In some embodiments, the Cpf1 protein is provided to the plant cells as a polypeptide, the polypeptide being taken up into the plant cell interior.

In other embodiments, the Cpf1 protein is provided by introducing into the plant cell a nucleic acid construct, i.e. a polynucleotide, for expression of the Cpf1 protein in the plant cell. Such nucleic acid construct may be any suitable construct known in the art and which is used to deliver exogenous DNA into a host cell with the purpose of expression in the host cell of a DNA region (here the Cpf1 gene) comprised on the construct.

Introduction of the Cpf1 protein or the nucleic acid construct encoding the same may be accomplished by any method known which permits the successful introduction of the protein or the nucleic acid construct into the plant cells, and which, in case of a nucleic acid construct, results in the expression of the introduced nucleic acid. Methods included but are not limited to such methods as transfection, microinjection, electroporation, nucleofection and lipofection.

The crRNA may also be introduced in the plant cell by any suitable method. For example, crRNA may be provided to the plant cell directly, or, in a preferred embodiment, by introducing into the plant cell a nucleic acid construct for expression of the crRNA in the plant cell.

Such nucleic acid construct may be any suitable construct known in the art and which is used to deliver exogenous DNA into a host cell with the purpose of expression in the host cell of a DNA region (here the crRNA) comprised on the construct.

Introduction of the crRNA or the nucleic acid construct encoding the same, may be accomplished by any method known which permits the successful introduction of the crRNA or the nucleic acid construct into the plant cells, and which, in case of a nucleic acid construct, results in the expression of the introduced crRNA. Methods included but are not limited to such methods as transfection, microinjection, electroporation, nucleofection and lipofection.

Preferably the Cpf1 protein and the crRNA are introduced in the plant cell using a nucleic acid construct for expression of both the Cpf1 protein and the crRNA in the plant cell. In such embodiment, it is however preferred that the nucleic acid sequence encoding the site-Cpf1 protein and the nucleic acid sequence encoding the crRNA are under control of different promoters.

For example, the crRNA may, preferably, be under control of, i.e. operably linked to, a pol Ill promoter (such as U6 and H1) preferably for expression in plant; RNA pol III promoters, such as U6 and H1, which are commonly used to express these small RNAs (see e.g. Ma et al. Molecular Therapy Nucleic Acids (2014) 3, e161).

For example, the Cpf1 protein may, preferably, be under control of a constitutive promotes, preferably for expression in plant, such as the 35 S promoter (e.g. the 35 S promoted from cauliflower mosaic virus (CaMV; Odell et al. Nature 313:810-812; 1985). Other suitable constitutive promoters include, but are not limited to, the cassava vein mosaic virus (CsVMV) promoter, and the sugarcane bacilliform badnavirus (ScBV) promoter (see e.g. Samac et al. Transgenic Res. 2004 August; 13(4):349-61.) Other constitutive promoters include, for example, the core promoter of the Rsyn7 promoter and other constitutive promoters disclosed in WO 99/43 838 and U.S. Pat. No. 6,072,050; ubiquitin (Christensen et al., Plant Mol. Biol. 12:619-632, 1989 and Christensen et al., Plant Mol. Biol. 18:675-689, 1992); pEMU (Last et al., Theor. Appl. Genet. 81:581-588, 1991); AA6 promoter (WO2007/069894); and the like.

The nucleic acid constructs may also include transcription termination regions. Where transcription termination regions are used, any termination region may be used in the preparation of the nucleic acid constructs.

In a preferred embodiment, the nucleic acid construct is for transient expression. In other words, the expression in the plant material is temporary as a consequence of the non-permanent presence of the nucleic acid construct. Expression may, for instance, be transient when the construct is not integrated into the host genome. For example, Cpf1 protein and crRNA may be transiently provided to a plant cell, followed by a decline in the amount of either or both of the components. Subsequently, the plant cell, progeny of the plant cell, and plants which comprise the plant cell or have been derived from the plant protoplast wherein the duplex DNA has been altered, comprise a reduced amount of either or both of the components used in the method of the invention, or no longer contain one or more of the components.

In conjunction with any of the methods and preferred embodiments as disclosed herein, the nucleic acid construct may be optimized for increased expression in the transformed plant.

In this embodiment, there is provided for the method of the invention, wherein the nucleic acid sequence encoding the Cpf1 protein is codon-optimized for expression in the plant cell. For instance, the nucleic acid sequence encoding the Cpf1 protein may be codon-optimized for expression in tomato, wherein said tomato preferably is Solanum lycopersicum.

That is, the nucleic acid construct encoding the Cpf1 protein can be synthesized using plant-preferred codons for improved expression. See, for example, Campbell and Gowri, (Plant Physiol. 92:1-11, 1990) for a discussion of host-preferred codon usage. Methods are available in the art for synthesizing plant-preferred genes (see, for example, Murray et al., Nucleic Acids Res. (1989) 17:477-498, or Lanza et al. (2014) BMC Systems Biology 8:33-43).

Introduction in the plant cell of the Cpf1 protein, the crRNA, and/or, where applicable, a nucleic acid construct encoding the Cpf1 protein and/or the crRNA, may be accomplished by any method known which permits the successful introduction thereof into the plant cell, and which, in case of a nucleic acid construct, results in the expression of the introduced Cpf1 protein and/or crRNA. Methods included but are not limited to such methods as transfection, microinjection, electroporation, nucleofection and lipofection.

However, in a preferred embodiment, there is provided for a method of the invention wherein in step (b) the duplex DNA is exposed to the Cpf1 protein and the crRNA, by introduction thereof, or of a nucleic acid construct encoding the same, in the plant cell or plant protoplast using polyethylene glycol mediated transformation.

Polyethylene glycol (PEG) is a polyether compound with many applications from industrial manufacturing to medicine. PEG is also known as polyethylene oxide (PEO) or polyoxyethylene (POE). The structure of PEG is commonly expressed as H—(O-CH2-CH2)n-OH. Preferably, the PEG used in the method according to the invention is an oligomer and/or polymers, or mixtures thereof with a molecular mass below 20,000 g/mol. PEG-mediated gene transformation has been known since 1985. The first method for plant protoplast transformation utilized PEG (Krens et al. (1982) Nature 296:72-74; Potyrykus et al. (1985) Plant Mol. Biol. Rep. 3:117-128; Negrutiu et al. (1987) Plant Mol.Biol. 8:363-373). The technique is applicable to protoplasts from many different plants (Rasmussen et al. (1993) Plant Sci. 89:199-207). PEG is thought to stimulate transformation by precipitating the DNA, in the presence of divalent cations, onto the surface of the plant protoplasts from where it then becomes internalized (Maas & Werr (1989) Plant Cell Rep. 8:148-151). In a preferred embodiment, a solution comprising 400 g/L PEG 4000 and 0.1M Ca(NO₃)₂ is used for transformation.

However, hitherto the use of PEG transformation in the method of the invention to introduce into the plant cell or plant protoplast the Cpf1 protein and/or the crRNA, or a nucleic acid construct encoding either or both of the Cpf1 protein and the crRNA has not been proposed.

In an embodiment the weight ratio of Cpf1 protein expressing plasmid relative to crRNA expressing plasmid is in the range of 0.1:20, such as 0.5-10, or 0.75:5, more preferred in the range of 1:3, even more preferred in the range of 1.5:2.5, such as about 1:2.

In an embodiment, at least 0.1 μg of Cpf1 expressing plasmid is used, such as at least 0.5 μg, at least 1 μg, at least 5 μg, or at least 10 μg.

In an embodiment, said at least 0.1 μg, such as at least 0.5 μg, at least 1 μg, at least 5 μg, or at least 10 μg, of Cpf1 expressing plasmid is combined with crRNA expressing plasmid in the appropriate weight range as taught herein, and said combination of Cpf1 expressing plasmid and crRNa expressing plasmid is combined with 10,000-10,000,000, preferably about 50,000-5,000,000, more preferably about 100,000-1,000,000, for example about 500,000 plant cells or plant protoplasts.

In an embodiment, the plant cells or plant protoplasts may be present in a volume of about 500 μl. In an embodiment, the volume ratio of plant cell/plant protoplast (including Cpf1 protein/crRNA) to PEG solution, which is preferably a solution comprising 400 g/L PEG 4000 and 0.1M Ca(NO₃)₂, is in the range of 1:0.5 to 1:1.5, and preferably about 1:1.

In an embodiment, PEG transfection may be allowed to take place for a period of time in the range of 10-60 minutes, such as 15-40 minutes, preferably about 20 minutes.

Subsequently, a 0.1-0.6 M, such as a 0.2-0.4 M, or about 0.275 M, Ca(NO₃)₂ solution may be added to the transfection solution taught above. Preferably about 8-12 times, such as about 10 times the volume of the transfection solution taught above may be used prior to harvesting by centrifugation.

According to another embodiment, there is provided for a method according to the invention wherein the method comprises a step of synchronizing the cell cycle of the plant cell, preferably before and/or during performing step (b), preferably wherein synchronizing is performed by contacting the plant cell with a synchronizing agent.

Such method of synchronizing the cell cycle of the plant cell has been described in detail in European patent EP2516652, incorporated herein by reference. More particular, synchronizing the plant cells, for example, the plant protoplasts may be advantageous in certain embodiments of the invention to further enhance efficacy of the introduction of the alteration in the duplex DNA. Thus, in certain embodiments, the method comprises a step of synchronizing the cell cycle of the plant cell.

Preferably, synchronization is performed before and/or during performing step (b). In case synchronization is performed before step (b), most of the plant cells will be in the same phase of the cell cycle when the duplex DNA is exposed to, for example, the Cpf1 protein and/or crRNA as defined herein. This may be advantageous and increase the rate of introduction of the alteration in the duplex DNA. Also in case the plant cells are synchronized during step (b), this may increase overall introduction of the alteration in the duplex DNA.

Synchronizing the plant cell may be accomplished by any suitable means. For example, synchronization of the cell cycle may be achieved by nutrient deprivation such as phosphate starvation, nitrate starvation, ion starvation, serum starvation, sucrose starvation, auxin starvation. Synchronization can also be achieved by adding a synchronizing agent to the plant cell.

Synchronizing agents such as aphidocolin, hydroxyurea, thymidine, colchicine, cobtorin, dinitroaniline, benefin, butralin, dinitramine, ethalfluralin, oryzalin, pendimethalin, trifluralin, amiprophos-methyl, butamiphos dithiopyr, thiazopyr propyzamide, tebutam DCPA (chlorthal-dimethyl), mimosine, anisomycin, alpha amanitin, lovastatin, jasmonic acid, abscisic acid, menadione, cryptogeine, hydrogenperoxide, sodiumpermanganate, indomethacin, epoxomycin, lactacystein, icrf 193, olomoucine, roscovitine, bohemine, staurosporine, K252a, okadaic acid, endothal, caffeine, MG 132, cycline dependent kinases and cycline dependent kinase inhibitors as well as their target mechanism, the amounts and concentrations and their associated cell cycle phase are described for instance in “Flow Cytometry with plant cells”, J. Dolezel c.s. Eds. Wiley-VCH Verlag 2007 pp 327 ff. There exists a preference for aphidicolin and/or hydroxyurea.

Preferably, in the method of the invention, synchronizing the cell cycle synchronizes the plant cell in the S-phase, the M-phase, the G1 and/or G2 phase of the cell cycle.

According to another preference, in the method of the invention two or more crRNA's are used. The two or more crRNA's may direct the Cpf1 protein to the same site in the DNA duplex, or to a different site (for example in order to introduce more than one double-strand break).

According to another preference the Cpf1 protein and the crRNA are transiently expressed in the plant cell, as already discussed herein above.

With the method taught herein an alteration in introduced in the duplex DNA in a plant protoplast. The targeted alteration may comprise the insertion, deletion or modification of at least one base pair. For example, the targeted alteration may comprise the deletion of at least one base pair and the insertion of at least one base pair. For example, 1, 2, 3, 4, 5, 6, 7, 8, 9, 10 or more base pairs may be altered with the method of the invention. More than one modification may be introduced in a single experiment, and/or the experiment may be repeated to introduce subsequent alterations in the duplex DNA in the plant cell.

According to another embodiment of the present invention, the method further comprises the step of regenerating a plant or descendent thereof comprising the targeted alteration. The skilled person is well aware of methods and protocols of regenerating a plant from a plant protoplast. Progeny, descendant's, variants, and mutants of the regenerated plants are also included within the scope of the invention, provided that these parts comprise the targeted alteration introduced with the method taught herein.

Finally, there is provided for a plant, plant part, seed, or plant cell obtained by the method of the invention, wherein the plant, plant part, seed, or plant cell is modified by comprising the targeted alteration when compared to a control plant, plant part, seed, or plant cell, and wherein the control plant, plant part, seed, or plant cell is a plant, plant part, seed, or plant cell before the targeted alteration was introduced by the method as taught herein.

Having now generally described the invention, the same will be more readily understood through reference to the following examples which is provided by way of illustration and is not intended to be limiting of the present invention.

### BRIEF DESCRIPTION OF THE FIGURES

FIG. 1A-1B shows the protein sequence of Cpf1-His-NLS, in which the 6×His domain is underlined and the NLS sequence is shown in bold, and the nucleotide sequence of the Cpf1-His-NLS ORF.

FIG. 2 shows the sequences of the crRNAs and sgRNAs used in the examples section. The sequence of the Arabidopsis thaliana U6 promoter is underlined. The sequences derived from the target genes are shown in bold and the remainder of the crRNA or sgRNA is shown in italics.

FIG. 3 shows quantification of the indels found in tomato protoplasts treated with either Cpf1 or Cas9. The Cpf1 crRNA1 and Cas9 sgRNA generate indels at the same target sequence whereas the Cpf1 crRNA2 creates indels at an alternative position. Control samples were derived from protoplasts transfected only with the Cpf1 or Cas9 expressing plasmid but not the plasmid expressing the corresponding targeting RNA.

FIG. 4 shows indel mutations generated by the Cpf1/crRNA1 PDS1 and the Cpf1/crRNA2 PDS1 reagents at the tomato PDS1 gene.

FIG. 5A-5B shows the genotyping of calli derived from protoplasts treated with Cpf1 protein and crRNA. Panel A: PCR products from calli derived from protoplasts treated with the Cpf1 protein and crRNA1 PDS1 and then digested with the restriction enzyme XhoI. This enzyme cuts in the middle of the PCR product, producing two digestion products of a very similar sizes that are not resolved on an agarose gel. Panel B: PCR products from calli derived from protoplasts treated with the Cpf1 protein and crRNA2 PDS1 and then digested with the restriction enzyme Sau3AI. This enzyme removes a 100 bps fragment from the PCR product.

### EXAMPLES

### Example 1

### Constructs

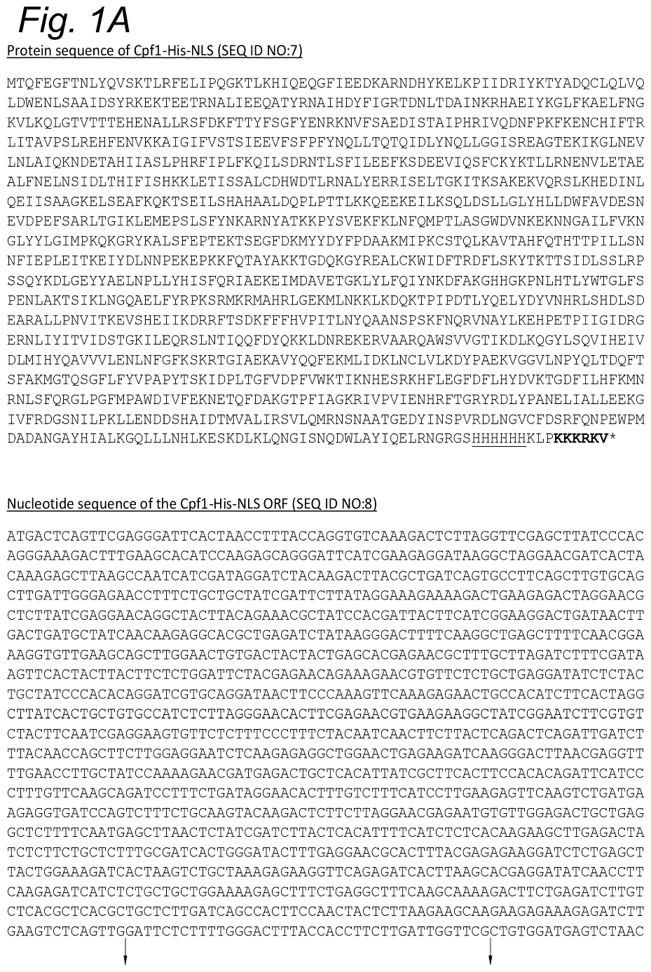

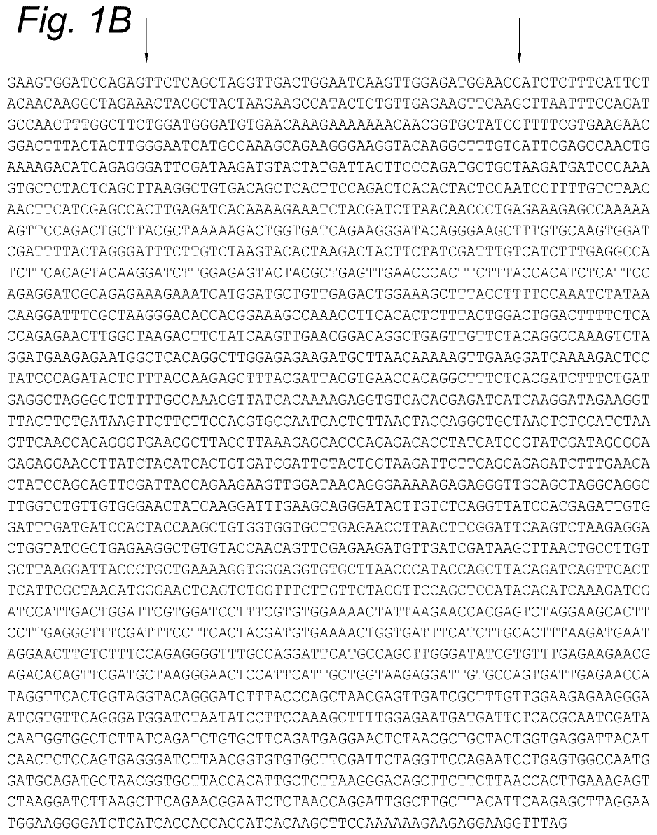

The Cpf1 ORF from the bacterium Acidaminococcus sp BV3L6 was codon optimized for expression in Solanum lycopersicum and a NLS and 6×His sequence was added to the C terminus (FIG. 1). This modified Cpf1 ORF was then synthesized (www.geneart.com) in the Gateway entry vector pDONR221. The Cpf1 ORF was then recombined with the plant expression vector K2GW7 in a Gateway LR reaction resulting in a vector (KG9858) where the Cpf1 ORF is expressed using the constitutive 35S promoter.

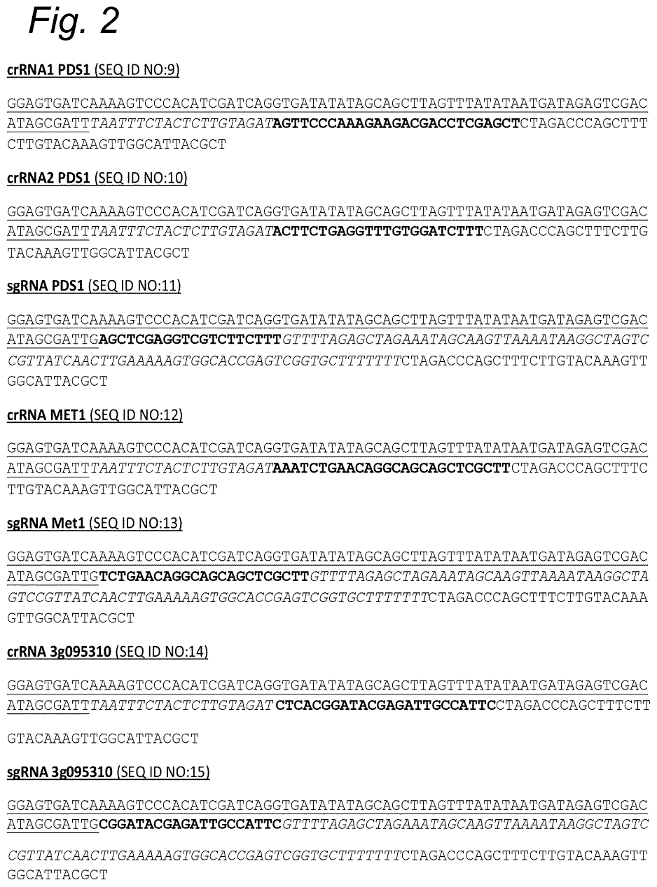

We designed four Cpf1 crRNAs (crRNAs) which will target the Cpf1 protein to three different genes in the tomato genome, PDS1, Met1 and Solyc3g095310. In order to compare the mutagenesis frequency of Cpf1 and Cas9 we also designed guide RNAs (sgRNAs) for Cas9 that will target the Cas9 protein to the same location. The crRNAs and sgRNAs were expressed using the U6 polIII promoter from Arabidopsis thaliana. The expression cassettes used in these experiments are shown in FIG. 2.

### Tomato Protoplast Isolation and Transfection

In vitro shoot cultures of Solanum lycopersicum var Moneyberg were maintained on MS20 medium with 0.8% agar in high plastic jars at 16/8 h photoperiod of 2000 lux at 25° C. and 60-70% RH. Young leaves (1 g) were gently sliced perpendicularly to the mid nerve to ease the penetration of the enzyme mixture. Sliced leaves were transferred to the enzyme mixture (2% Cellulase Onozuka RS, 0.4% Macerozyme Onozuka R10 in CPW9M) and cell wall digestion was allowed to proceed overnight in the dark at 25° C. The protoplasts were filtered through a 50 μm nylon sieve and were harvested by centrifugation for 5 minutes at 800 rpm. Protoplasts were resuspended in CPW9M (Frearson, 1973) medium and 3 mL CPW18S (Frearson, 1973) was added at the bottom of each tube using a long-neck glass Pasteur pipette. Live protoplasts were harvested by centrifugation for 10 minutes at 800 rpm as the cell fraction at the interface between the sucrose and CPW9M medium. Protoplasts were counted and resuspended in MaMg (Negrutiu, 1987) medium at a final density of 10⁶ per mL.

For the protoplast transfections 10 μg of the Cas9 or Cpf1 expression plasmids and 20 μg of the sgRNA or crRNA expressing plasmids were mixed with 500 μL (500000 protoplasts) of the protoplast suspension and 500 μL of PEG solution (400 g/l poly(ethylene glycol) 4000, Sigma-Aldrich #81240; 0.1M Ca(NO₃)₂) was then added and the transfection was allowed to take place for 20 minutes at room temperature. Then, 10 mL of 0.275 M Ca(NO₃)₂ solution was added and thoroughly, but gently mixed in. The protoplasts were harvested by centrifugation for 5 minutes at 800 rpm and resuspended in 9M culture medium at a density of 0.5×10⁶ per ml and transferred to a 4 cm diameter petri dish and an equal volume of 2% alginate solution (20 g/l Alginate-Na (Sigma-Aldrich #A0682), 0.14 g/l CaCl₂·2H₂O, 90 g/l mannitol) was added. Then 1 ml aliquots (125000 transfected protoplasts) were spread over Ca-Agar plates (72.5 g/l mannitol, 7.35 g/l CaCl₂·2H₂O, 8 g/l agar, pH5.8) and allowed to polymerise for 1 hour. For protoplast cultivation the embedded protoplasts were grown in a 4 cm tissue culture dish containing 4 ml of K8p (Kao, 1975) culture medium. To detect indels in tomato protoplasts the disc of transfected protoplasts was removed from the dish after 48 hours and the alginate was dissolved (which buffer) and the protoplasts were isolated. For the regeneration of calli, the protoplasts were incubated in the K8p medium for 21 days at 28° C. in the dark. After this period the discs of transfected protoplasts were transferred to solid GM medium (Tan, 1987) supplemented with 1 mg·l⁻¹ zeatin and 0.2 mg·l⁻¹ GA3 and grown for a further 3 weeks at which point the calli were approximately 0.3 mm in size. The aliginate was then dissolved and the calli were spread on a fresh plate of GM medium and allowed to grow until they were approximately 1.5 mm, at which point they were once again transferred to fresh medium and then genotyped after a further 14 days.

### Genotyping Protoplasts and Calli

Tomato protoplasts that had been transfected with the Cas9/sgRNA plasmids or the Cpf1/crRNA plasmids were cultivated for 48 hours, the alginate dissolved, and collected by centrifugation. Total genomic DNA (gDNA) was then isolated from each sample using the DNeasy Plant Mini Kit (Qiagen). This gDNA was then used in a PCR reaction to amplify the target sites. The following primers were used to amplify the different target sites: SIPDS1, 5′-TGTGCAGAACCACTCCCT-3′ and 5′-TTTAGTTGGGCGCGGAGA-3′; Solycl3g095310, 5′-ATGGGAAGCGGTGAAAGAAAG-3′ and 5′-AGGGTCACGATGAAGAGTTGG-3′; SIMET1, 5′-GGACACAAAAAGAACAAACGCA-3′ and 5′-TATGAACCCGCCCTGAGT-3′. These were then used to generate a library which was then sequenced on the MiSeq sequencer with each sample identified using a unique 5 bp tag. After sequencing the reads of each sample were processed to identify the number and types of indels present in each sample. Calli were genotyped directly using the direct PCR kit (Phire Plant Direct PCR kit, Thermo Scientific) and the gene specific primers described above. The resulting PCR products were then sequenced to identify which calli contained INDEL mutations at the intended target site. These were then transferred to MS medium supplemented with 2 mg·l⁻¹ zeatin and 0.1 mg·l⁻¹ IAA media after which regenerated tomato plantlets were rooted on MS medium supplemented with 0.5 mg·l⁻¹ IBA before transfer to the greenhouse.

### RESULTS

We tested the ability of Cpf1 to generate INDEL mutations in the genome plant cells by expressing the Cpf1 protein and its crRNA ectopically in tomato protoplasts. First a Cpf1 ORF optimized for codon usage in tomato was constructed together with a nuclear localization signal (NLS) fused at the C terminus. This was then cloned behind the constitutive CaMV 35S promoter for expression in plant cells. This vector was then introduced into purified tomato leaf protoplasts together with plasmid expressing a crRNA driven by the Arabidopsis thaliana U6 promoter. In such a system the Cpf1 mRNA and the crRNA will be expressed at high levels for a short period, 24-48 hours, at which point the introduced plasmids will become degraded by cellular nucleases and the Cpf1 reagents will disappear from the cell. While they are present they are able to find the specific target site in the genome at create INDEL mutations. The introduced plasmids rarely integrate into the genome and so this approach does not result in transgenic lines. The protoplasts were then cultured for 48 hours and then destroyed and genotyped for the presence of an INDEL at the target site.

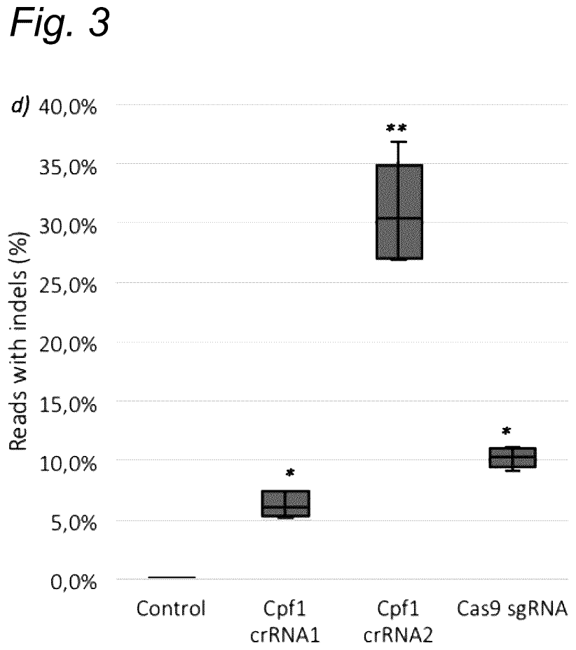

We found that the Cpf1 protein, when expressed together with either of the crRNAs targeting the SIPDS1 gene, was able to produce INDEL mutations at a high efficiency in tomato protoplasts (FIG. 3). Each transfection was performed in triplo and the samples were sequenced separately to test the effects of Cpf1 in different biological samples. When the crRNA2 PDS1 was used, 30.7% of the reads contained an INDEL at the target site. In contrast, for crRNA1 PDS1 approximately 6.5% of the sequence reads contained INDELs. This result demonstrates that the Cpf1 protein is very efficient at generating INDELs in plant cells and that the efficiency varies depending on the target sequence. As the sgRNA PDS1 targets the same sequence as crRNA PDS1, this allows us to make a direct comparison between the mutation efficiencies obtained when using either the Cas9 or the Cpf1 protein so that we can assess which works better in plant cells. In the sequence reads generated from the SpCas9/sgRNA PDS1 samples, 10% contained INDELs. This is not significantly different from the efficiency obtained using crRNA1 PDS1 as so we can conclude that 48 hrs after transfection both CRISPR proteins had generated mutations at similar frequencies.

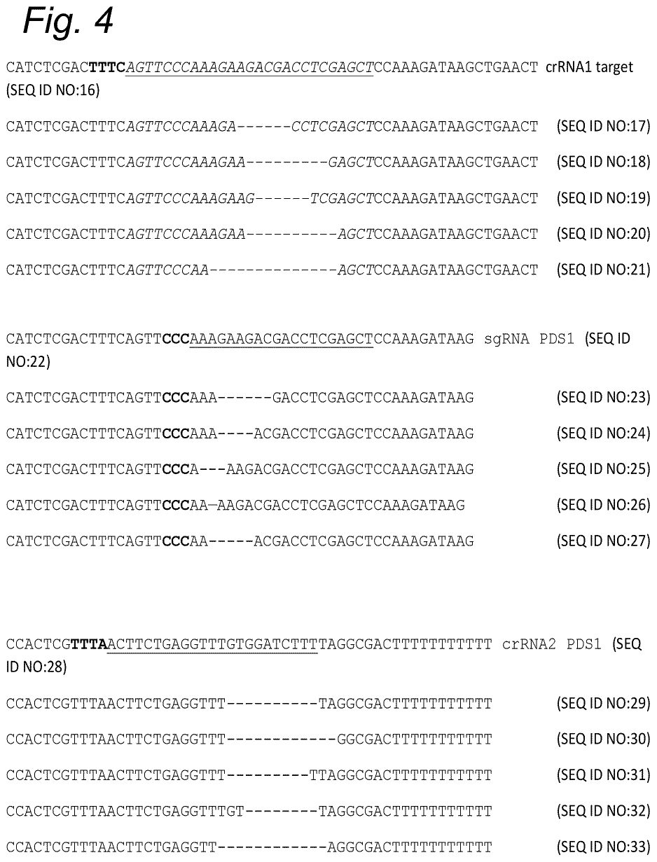

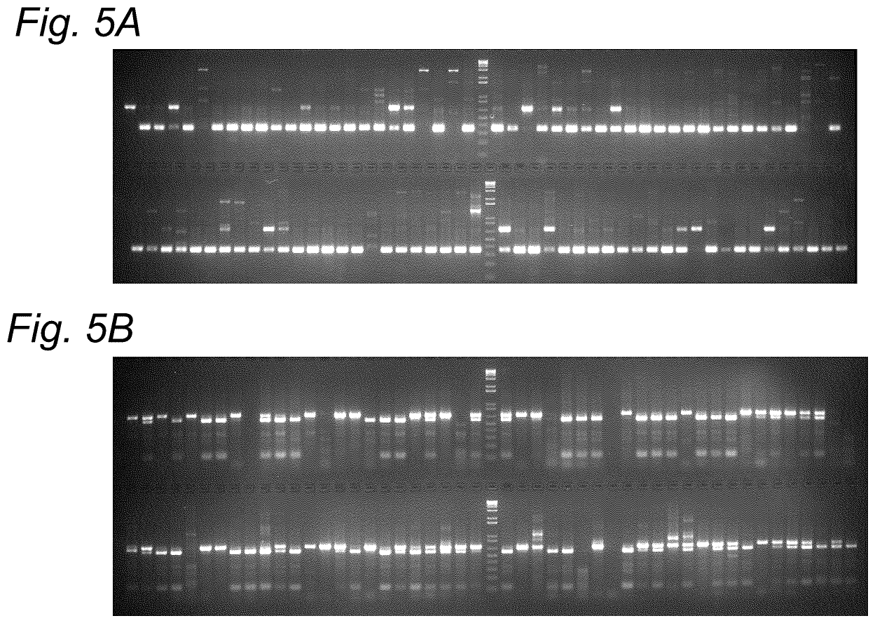

If Cpf1 is to be used to produce novel plants with improved growth and yield characteristics then it is important to demonstrate that plants carrying INDELs at the target sequence can be regenerated from mutated protoplasts. Therefore, we repeated the tomato protoplast transfections described above but in this case maintained the cells in growth medium to promote cell division until the protoplasts had formed calli large enough that could be sampled and genotyped. Each individual callus was sampled by the removal of a small amount of tissue that was used as a template in a direct PCR reaction together with primers designed to amplify the target sites in the PDS1 gene. The Cpf1 target sites both contain a restriction site (XhoI for crRNA1 PDS1 and Sau3AI for crRNA2 PDS1) which were lost when INDELs were created (FIGS. 3 and 4). Therefore, calli containing INDELs could be easily identified by digesting the PDS1 PCR product with the appropriate restriction enzyme and analysis of these on agarose gels. Calli containing an INDEL mutation gave PCR products that were resistant to restriction enzyme digestion and in this way we were able to easily quantify the mutants. We found that at the crRNA1 PDS1 target, 32% of the calli contained INDELs (FIG. 5), approximately five fold higher than estimated using the sequencing approach. We also found more mutant calli than expected when the crRNA2 PDS1 target was analyzed (FIG. 5). In this case, 57% of the calli contained INDELs at the target. Therefore we were able to conclude that the transient expression of the Cpf1 protein and its crRNA does not negatively influence protoplast growth and that the mutant protoplasts are able to regenerate to form mutant calli. It is very interesting to note that the mutagenesis efficiency in the calli was 2-5 fold higher than we had expected based upon the sequencing results. This is not observed when SpCas9 is used for mutagenesis in tomato protoplasts, where we do not find any significant difference between protoplast and callus efficiencies. One explanation for this may be that the Cpf1 protein and/or its CRNA are more stable in plant protoplasts compared with SpCas9 and/or its sgRNA. As a result of this the Cpf1 protein and crRNA will be active in the plant cells for a longer period and will therefore have a higher likelihood of inducing mutations.

We were also able to quantify the number of calli that contained biallelic mutations at the target sites. Obtaining biallelic mutants can be advantageous as the regenerated plants are usually immediately homozygous for the null alleles and do not have to go through a generation to selfing before they can be analyzed. This is of particular value in crop species that are vegetatively reproduced where mutations cannot be made homozygous. Calli derived from protoplasts transfected with the 35S::Cpf1 and crRNA1 PDS1 vectors showed biallelic mutations at a similar frequency to that found previously using SpCas9 (4/31 mutant calli contained biallelic mutations, 12%). However, we found a much higher percentage of biallelic mutations in calli derived from protoplasts transfected with the 35S::Cpf1 and crRNA2 PDS1 vectors (58/111 mutant calli contained biallelic mutations, 52%). These results demonstrate that Cpf1 is significantly better than SpCas9 for mutagenesis in plant cells. Such a high level of biallelic mutation formation has not previously been reported in plant cells and also represents a significant breakthrough for the mutagenesis of polyploidy crop species. The mutant calli were maintained on growth medium until they regenerated shoots. These were then genotyped to confirm that the mutations present in the callus were also present in the regenerated shoots. We always found the same mutation in the shoots as was originally present in the callus. Therefore, we have been able to show that the Cpf1 CRISPR system can be used to produce mutant plants.

To test whether the Cpf1 CRISPR system was able to generate mutations at other loci in the tomato genome we performed additional similar experiments at target sites in the SIMET1 and Solyc3g095310 loci. For both of these loci we found that the Cpf1 CRISPR system gave significantly higher mutagenesis frequencies than SpCas9. The mutagenesis frequency using Cpf1 was at least two fold higher than when SpCas9 was used. Therefore we conclude that in protoplasts of a polyploid species that the Cpf1 protein, due to its higher mutagenesis efficiency, would be able to introduce INDELs in more of the gene copies and consequently would be more suitable than SpCas9 in producing plants with improved yield and nutritional qualities.

1.-16. (canceled)

17. A method for producing plant calli of the genus Solanum,
wherein the produced plant calli have a targeted alteration in a target sequence that is comprised within two or more gene copies, the method comprising:
(a) providing plant protoplasts comprising said target sequence, wherein said plant protoplasts are of the genus Solanum;
(b) exposing said target sequence in the provided protoplasts to:
a single RNA-guided endonuclease Cpf1 protein; and
a crRNA comprising a guide sequence for targeting said Cpf1 protein to the target sequence comprised within the two or more gene copies, by introducing into the plant protoplasts said Cpf1 protein, or nucleic acid encoding the same, and said crRNA, or nucleic acid encoding the same, using polyethylene glycol and divalent cation mediated transformation, resulting in one targeted alteration of the two or more gene copies by said crRNA;
(c) regenerating calli from said protoplasts obtained in (b);
(d) selecting the calli comprising the targeted alteration of the two or more gene copies, wherein the Cpf1 protein comprises an amino acid sequence having at least 99% sequence identity to SEQ ID NO: 7.

18. The method of claim 17, further comprising a step of synchronizing the cell cycle phase of the protoplast, preferably before and/or during performing step (b), preferably wherein synchronizing is performed by contacting the protoplast with a synchronizing agent.

19. The method of claim 18, wherein the step of synchronizing the cell phase synchronizes the protoplast in the S-phase, the M-phase, the G1 and/or G2 phase of the cell cycle.

20. The method of claim 17, wherein the alteration comprises the insertion, deletion or modification of at least one base pair.

21. The method of claim 17, wherein the alteration comprises the deletion of at least one base pair and the insertion of at least one base pair.

22. The method of claim 17, wherein the targeted alteration is biallelic.

23. The method of claim 17, further comprising a step of regenerating a plant of the genus Solanum from the calli, wherein said plant or progeny thereof comprises the targeted alteration.

24. The method of claim 17, wherein said polyethylene glycol and divalent cation are present in a solution wherein the divalent cation is calcium.

25. The method of claim 24, wherein the polyethylene glycol and calcium are present in a solution wherein the calcium is provided as calcium nitrate.

26. The method of claim 25, wherein the polyethylene glycol and calcium nitrate are present in a solution wherein the concentration of calcium nitrate is 0.1-0.6M.

26. The method of claim 17, wherein the polyethylene glycol is PEG 4000.

27. The method of claim 17, further comprising a step of adding an alginate solution to the protoplasts after transformation.

28. The method of claim 17, wherein the targeted alteration results in null alleles and wherein the selected calli is homozygous for said null alleles.


SEQUENCE LISTING (33 sequences)

SEQ ID NO: 1 - DNA, 18 bp, synthetic construct
  misc_feature 1..18: note = First primer SlPDS1
  source 1..18: mol_type = other DNA
tgtgcagaaccactccct

SEQ ID NO: 2 - DNA, 18 bp, synthetic construct
  misc_feature 1..18: note = Second primer SlPDS1
  source 1..18: mol_type = other DNA
tttagttgggcgcggaga

SEQ ID NO: 3 - DNA, 21 bp, synthetic construct
  misc_feature 1..21: note = Primer 1 Solycl3g095310
  source 1..21: mol_type = other DNA
atgggaagcggtgaaagaaag

SEQ ID NO: 4 - DNA, 21 bp, synthetic construct
  misc_feature 1..21: note = Primer 2 Solycl3g095310
  source 1..21: mol_type = other DNA
agggtcacgatgaagagttgg

SEQ ID NO: 5 - DNA, 22 bp, synthetic construct
  misc_feature 1..22: note = Primer 1 SlMET1
  source 1..22: mol_type = other DNA
ggacacaaaaagaacaaacgca

SEQ ID NO: 6 - DNA, 18 bp, synthetic construct
  misc_feature 1..18: note = Primer 2 SlMET1
  source 1..18: mol_type = other DNA
tatgaacccgccctgagt

SEQ ID NO: 7 - protein, 1326 aa, synthetic construct
  REGION 1..1326: note = Protein sequence of Cpf1-His-NLS
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRGSHHHHHHKLPKKKRKV

SEQ ID NO: 8 - DNA, 3981 bp, synthetic construct
  misc_feature 1..3981: note = Nucleotide sequence of the Cpf1-His-NLS ORF
  source 1..3981: mol_type = other DNA
atgactcagttcgagggattcactaacctttaccaggtgtcaaagactcttaggttcgagcttatcccacagggaaagactttgaagcacatccaagagcagggattcatcgaagaggataaggctaggaacgatcactacaaagagcttaagccaatcatcgataggatctacaagacttacgctgatcagtgccttcagcttgtgcagcttgattgggagaacctttctgctgctatcgattcttataggaaagaaaagactgaagagactaggaacgctcttatcgaggaacaggctacttacagaaacgctatccacgattacttcatcggaaggactgataacttgactgatgctatcaacaagaggcacgctgagatctataagggacttttcaaggctgagcttttcaacggaaaggtgttgaagcagcttggaactgtgactactactgagcacgagaacgctttgcttagatctttcgataagttcactacttacttctctggattctacgagaacagaaagaacgtgttctctgctgaggatatctctactgctatcccacacaggatcgtgcaggataacttcccaaagttcaaagagaactgccacatcttcactaggcttatcactgctgtgccatctcttagggaacacttcgagaacgtgaagaaggctatcggaatcttcgtgtctacttcaatcgaggaagtgttctctttccctttctacaatcaacttcttactcagactcagattgatctttacaaccagcttcttggaggaatctcaagagaggctggaactgagaagatcaagggacttaacgaggttttgaaccttgctatccaaaagaacgatgagactgctcacattatcgcttcacttccacacagattcatccctttgttcaagcagatcctttctgataggaacactttgtctttcatccttgaagagttcaagtctgatgaagaggtgatccagtctttctgcaagtacaagactcttcttaggaacgagaatgtgttggagactgctgaggctcttttcaatgagcttaactctatcgatcttactcacattttcatctctcacaagaagcttgagactatctcttctgctctttgcgatcactgggatactttgaggaacgcactttacgagagaaggatctctgagcttactggaaagatcactaagtctgctaaagagaaggttcagagatcacttaagcacgaggatatcaaccttcaagagatcatctctgctgctggaaaagagctttctgaggctttcaagcaaaagacttctgagatcttgtctcacgctcacgctgctcttgatcagccacttccaactactcttaagaagcaagaagagaaagagatcttgaagtctcagttggattctcttttgggactttaccaccttcttgattggttcgctgtggatgagtctaacgaagtggatccagagttctcagctaggttgactggaatcaagttggagatggaaccatctctttcattctacaacaaggctagaaactacgctactaagaagccatactctgttgagaagttcaagcttaatttccagatgccaactttggcttctggatgggatgtgaacaaagaaaaaaacaacggtgctatccttttcgtgaagaacggactttactacttgggaatcatgccaaagcagaagggaaggtacaaggctttgtcattcgagccaactgaaaagacatcagagggattcgataagatgtactatgattacttcccagatgctgctaagatgatcccaaagtgctctactcagcttaaggctgtgacagctcacttccagactcacactactccaatccttttgtctaacaacttcatcgagccacttgagatcacaaaagaaatctacgatcttaacaaccctgagaaagagccaaaaaagttccagactgcttacgctaaaaagactggtgatcagaagggatacagggaagctttgtgcaagtggatcgattttactagggatttcttgtctaagtacactaagactacttctatcgatttgtcatctttgaggccatcttcacagtacaaggatcttggagagtactacgctgagttgaacccacttctttaccacatctcattccagaggatcgcagagaaagaaatcatggatgctgttgagactggaaagctttaccttttccaaatctataacaaggatttcgctaagggacaccacggaaagccaaaccttcacactctttactggactggacttttctcaccagagaacttggctaagacttctatcaagttgaacggacaggctgagttgttctacaggccaaagtctaggatgaagagaatggctcacaggcttggagagaagatgcttaacaaaaagttgaaggatcaaaagactcctatcccagatactctttaccaagagctttacgattacgtgaaccacaggctttctcacgatctttctgatgaggctagggctcttttgccaaacgttatcacaaaagaggtgtcacacgagatcatcaaggatagaaggtttacttctgataagttcttcttccacgtgccaatcactcttaactaccaggctgctaactctccatctaagttcaaccagagggtgaacgcttaccttaaagagcacccagagacacctatcatcggtatcgataggggagagaggaaccttatctacatcactgtgatcgattctactggtaagattcttgagcagagatctttgaacactatccagcagttcgattaccagaagaagttggataacagggaaaaagagagggttgcagctaggcaggcttggtctgttgtgggaactatcaaggatttgaagcagggatacttgtctcaggttatccacgagattgtggatttgatgatccactaccaagctgtggtggtgcttgagaaccttaacttcggattcaagtctaagaggactggtatcgctgagaaggctgtgtaccaacagttcgagaagatgttgatcgataagcttaactgccttgtgcttaaggattaccctgctgaaaaggtgggaggtgtgcttaacccataccagcttacagatcagttcacttcattcgctaagatgggaactcagtctggtttcttgttctacgttccagctccatacacatcaaagatcgatccattgactggattcgtggatcctttcgtgtggaaaactattaagaaccacgagtctaggaagcacttccttgagggtttcgatttccttcactacgatgtgaaaactggtgatttcatcttgcactttaagatgaataggaacttgtctttccagaggggtttgccaggattcatgccagcttgggatatcgtgtttgagaagaacgagacacagttcgatgctaagggaactccattcattgctggtaagaggattgtgccagtgattgagaaccataggttcactggtaggtacagggatctttacccagctaacgagttgatcgctttgttggaagagaagggaatcgtgttcagggatggatctaatatccttccaaagcttttggagaatgatgattctcacgcaatcgatacaatggtggctcttatcagatctgtgcttcagatgaggaactctaacgctgctactggtgaggattacatcaactctccagtgagggatcttaacggtgtgtgcttcgattctaggttccagaatcctgagtggccaatggatgcagatgctaacggtgcttaccacattgctcttaagggacagcttcttcttaaccacttgaaagagtctaaggatcttaagcttcagaacggaatctctaaccaggattggcttgcttacattcaagagcttaggaatggaaggggatctcatcaccaccaccatcacaagcttccaaaaaagaagaggaaggtttag

SEQ ID NO: 9 - DNA, 164 bp, synthetic construct
  misc_feature 1..164: note = crRNA1 PDS1
  source 1..164: mol_type = other DNA
ggagtgatcaaaagtcccacatcgatcaggtgatatatagcagcttagtttatataatgatagagtcgacatagcgatttaatttctactcttgtagatagttcccaaagaagacgacctcgagctctagacccagctttcttgtacaaagttggcattacgct

SEQ ID NO: 10 - DNA, 160 bp, synthetic construct
  misc_feature 1..160: note = crRNA2 PDS1
  source 1..160: mol_type = other DNA
ggagtgatcaaaagtcccacatcgatcaggtgatatatagcagcttagtttatataatgatagagtcgacatagcgatttaatttctactcttgtagatacttctgaggtttgtggatctttctagacccagctttcttgtacaaagttggcattacgct

SEQ ID NO: 11 - DNA, 221 bp, synthetic construct
  misc_feature 1..221: note = sgRNA PDS1
  source 1..221: mol_type = other DNA
ggagtgatcaaaagtcccacatcgatcaggtgatatatagcagcttagtttatataatgatagagtcgacatagcgattgagctcgaggtcgtcttctttgttttagagctagaaatagcaagttaaaataaggctagtccgttatcaacttgaaaaagtggcaccgagtcggtgctttttttctagacccagctttcttgtacaaagttggcattacgct

SEQ ID NO: 12 - DNA, 163 bp, synthetic construct
  misc_feature 1..163: note = crRNA MET1
  source 1..163: mol_type = other DNA
ggagtgatcaaaagtcccacatcgatcaggtgatatatagcagcttagtttatataatgatagagtcgacatagcgatttaatttctactcttgtagataaatctgaacaggcagcagctcgcttctagacccagctttcttgtacaaagttggcattacgct

SEQ ID NO: 13 - DNA, 224 bp, synthetic construct
  misc_feature 1..224: note = sgRNA Met1
  source 1..224: mol_type = other DNA
ggagtgatcaaaagtcccacatcgatcaggtgatatatagcagcttagtttatataatgatagagtcgacatagcgattgtctgaacaggcagcagctcgcttgttttagagctagaaatagcaagttaaaataaggctagtccgttatcaacttgaaaaagtggcaccgagtcggtgctttttttctagacccagctttcttgtacaaagttggcattacgct

SEQ ID NO: 14 - DNA, 161 bp, synthetic construct
  misc_feature 1..161: note = crRNA 3g095310
  source 1..161: mol_type = other DNA
ggagtgatcaaaagtcccacatcgatcaggtgatatatagcagcttagtttatataatgatagagtcgacatagcgatttaatttctactcttgtagatctcacggatacgagattgccattcctagacccagctttcttgtacaaagttggcattacgct

SEQ ID NO: 15 - DNA, 221 bp, synthetic construct
  misc_feature 1..221: note = sgRNA 3g095310
  source 1..221: mol_type = other DNA
ggagtgatcaaaagtcccacatcgatcaggtgatatatagcagcttagtttatataatgatagagtcgacatagcgattgcggatacgagattgccattcgttttagagctagaaatagcaagttaaaataaggctagtccgttatcaacttgaaaaagtggcaccgagtcggtgctttttttctagacccagctttcttgtacaaagttggcattacgct

SEQ ID NO: 16 - DNA, 58 bp, synthetic construct
  misc_feature 1..58: note = crRNA1 target
  source 1..58: mol_type = other DNA
catctcgactttcagttcccaaagaagacgacctcgagctccaaagataagctgaact

SEQ ID NO: 17 - DNA, 52 bp, synthetic construct
  misc_feature 1..52: note = Indel mutation generated by the Cpf1/crRNA1 PDS1
  source 1..52: mol_type = other DNA
catctcgactttcagttcccaaagacctcgagctccaaagataagctgaact

SEQ ID NO: 18 - DNA, 49 bp, synthetic construct
  misc_feature 1..49: note = Indel mutation 2 generated by the Cpf1/crRNA1 PDS1
  source 1..49: mol_type = other DNA
catctcgactttcagttcccaaagaagagctccaaagataagctgaact

SEQ ID NO: 19 - DNA, 52 bp, synthetic construct
  misc_feature 1..52: note = Indel mutation 3 generated by the Cpf1/crRNA1 PDS1
  source 1..52: mol_type = other DNA
catctcgactttcagttcccaaagaagtcgagctccaaagataagctgaact

SEQ ID NO: 20 - DNA, 48 bp, synthetic construct
  misc_feature 1..48: note = Indel mutation 4 generated by the Cpf1/crRNA1 PDS1
  source 1..48: mol_type = other DNA
catctcgactttcagttcccaaagaaagctccaaagataagctgaact

SEQ ID NO: 21 - DNA, 44 bp, synthetic construct
  misc_feature 1..44: note = Indel mutation 5 generated by the Cpf1/crRNA1 PDS1
  source 1..44: mol_type = other DNA
catctcgactttcagttcccaaagctccaaagataagctgaact

SEQ ID NO: 22 - DNA, 51 bp, synthetic construct
  misc_feature 1..51: note = sgRNA PDS1
  source 1..51: mol_type = other DNA
catctcgactttcagttcccaaagaagacgacctcgagctccaaagataag

SEQ ID NO: 23 - DNA, 45 bp, synthetic construct
  misc_feature 1..45: note = Mutation 1 generated by Cas9/sgRNA PDS1
  source 1..45: mol_type = other DNA
catctcgactttcagttcccaaagacctcgagctccaaagataag

SEQ ID NO: 24 - DNA, 47 bp, synthetic construct
  misc_feature 1..47: note = Mutation 2 generated by Cas9/sgRNA PDS1
  source 1..47: mol_type = other DNA
catctcgactttcagttcccaaaacgacctcgagctccaaagataag

SEQ ID NO: 25 - DNA, 48 bp, synthetic construct
  misc_feature 1..48: note = Mutation 3 generated by the Cas9/sgRNA PDS1
  source 1..48: mol_type = other DNA
catctcgactttcagttcccaaagacgacctcgagctccaaagataag

SEQ ID NO: 26 - DNA, 49 bp, synthetic construct
  misc_feature 1..49: note = Mutation 4 generated by the Cas9/sgRNA PDS1
  source 1..49: mol_type = other DNA
catctcgactttcagttcccaaaagacgacctcgagctccaaagataag

SEQ ID NO: 27 - DNA, 46 bp, synthetic construct
  misc_feature 1..46: note = Mutation 5 generated by the Cas9/sgRNA PDS1
  source 1..46: mol_type = other DNA
catctcgactttcagttcccaaacgacctcgagctccaaagataag

SEQ ID NO: 28 - DNA, 53 bp, synthetic construct
  misc_feature 1..53: note = crRNA2 PDS1
  source 1..53: mol_type = other DNA
ccactcgtttaacttctgaggtttgtggatcttttaggcgacttttttttttt

SEQ ID NO: 29 - DNA, 43 bp, synthetic construct
  misc_feature 1..43: note = Indel mutation 1 generated by the Cpf1/crRNA2 PDS1
  source 1..43: mol_type = other DNA
ccactcgtttaacttctgaggttttaggcgacttttttttttt

SEQ ID NO: 30 - DNA, 41 bp, synthetic construct
  misc_feature 1..41: note = Indel mutation 2 generated by the Cpf1/crRNA2 PDS1
  source 1..41: mol_type = other DNA
ccactcgtttaacttctgaggtttggcgacttttttttttt

SEQ ID NO: 31 - DNA, 44 bp, synthetic construct
  misc_feature 1..44: note = Indel mutation 3 generated by the Cpf1/crRNA2 PDS1
  source 1..44: mol_type = other DNA
ccactcgtttaacttctgaggtttttaggcgacttttttttttt

SEQ ID NO: 32 - DNA, 45 bp, synthetic construct
  misc_feature 1..45: note = Indel mutation 4 generated by the Cpf1/crRNA2 PDS1
  source 1..45: mol_type = other DNA
ccactcgtttaacttctgaggtttgttaggcgacttttttttttt

SEQ ID NO: 33 - DNA, 41 bp, synthetic construct
  misc_feature 1..41: note = Indel mutation 5 generated by the Cpf1/crRNA2 PDS1
  source 1..41: mol_type = other DNA
ccactcgtttaacttctgaggttaggcgacttttttttttt